(19) United States
(12) Patent Application Publication
(10) Pub. No.: US 2023/0263563 A1
(43) Pub. Date: Aug. 24, 2023

(54) WATER TRAP, SMOKE EVACUATION TUBE SET, SURGICAL SYSTEM

(71) Applicant: KYOCERA Corporation, Kyoto-shi, Kyoto (JP)

(72) Inventors: Hiroaki YOSHIKAWA, Ritto-shi, Shiga (JP); Yukiko SHIMAUCHI, Takatsuki-shi, Osaka (JP)

(21) Appl. No.: 18/017,134

(22) PCT Filed: Jul. 6, 2021
(86) PCT No.: PCT/JP2021/025381

§ 371 Date: Jan. 20, 2023

(30) Foreign Application Priority Data
Jul. 22, 2020 (JP) 2020-125778

(51) Int. Cl. A61B 18/00

(52) U.S. Cl. CPC A61B 18/00; A61B 2218/008; A61B 2018/00595

(57) ABSTRACT

A medical water trap 20 includes a container 22, a partition wall 40, an inner cylinder 28, and a projecting portion 44. An intake port 34 and a discharge port 36 are formed in the container. The partition wall divides an internal space 38 of the container into a space 38A on the intake port side and a space 38B on the discharge port side. The inner cylinder communicates with the intake port and extends toward the partition wall in a space on the intake port side. The projecting portion is formed to project from the partition wall toward the intake port side and surround the inner cylinder when the partition wall is viewed from the intake port side in a plan view. At least one opening 42 is formed in the partition wall on a side closer to the wall surface of the container than the projecting portion.

### TECHNICAL FIELD

The present disclosure relates to a medical water trap.

### BACKGROUND OF INVENTION

In a laparoscopic surgery using a medical cautery instrument, surgical smoke generated in an abdominal cavity of a patient is sucked by a suction device and removed from the abdominal cavity. Here, in order to suppress moisture contained in the surgical smoke from flowing into the suction device, a water trap may be provided in a smoke evacuation channel for the surgical smoke.

### SUMMARY

A medical water trap according to the present disclosure includes: a container including an intake port and a discharge port; a partition wall configured to divide an internal space of the container into a space on the intake port side and a space on the discharge port side; an inner cylinder configured to communicate with the intake port and extending toward the partition wall in a space on the intake port side; and a projecting portion formed to project from the partition wall toward the intake port side and surround the inner cylinder in a plan view of the partition wall from the intake port side, wherein at least one opening is formed in the partition wall on a side closer to a wall surface side of the container than the projecting portion.

### BRIEF DESCRIPTION OF THE DRAWINGS

FIG. 1 is a schematic side cross-sectional view of a water trap according to a first embodiment of the present disclosure.

FIG. 2 is a schematic view illustrating a surgical system according to the first embodiment of the present disclosure.

FIG. 3 is a schematic perspective view illustrating an outer shape of the water trap according to the first embodiment of the present disclosure.

FIG. 4 is a schematic cross-sectional view taken along line A-A of the water trap in a direction of arrow illustrated in FIG. 1 when viewed from an intake port side.

FIG. 5 is a schematic side cross-sectional view of a water trap according to a comparative embodiment of the present disclosure.

FIG. 6 is a schematic side cross-sectional view of a water trap according to a second embodiment of the present disclosure.

FIG. 7 is a schematic side cross-sectional view of a water trap according to a third embodiment of the present disclosure.

### DESCRIPTION OF EMBODIMENTS

### First Embodiment

Embodiments according to the present disclosure will be described below with reference to the drawings. The drawings used in the following description are schematic views, and the dimension ratio of each member in the drawings is not strictly represented.

### Surgical System and Smoke Evacuation Tube Set

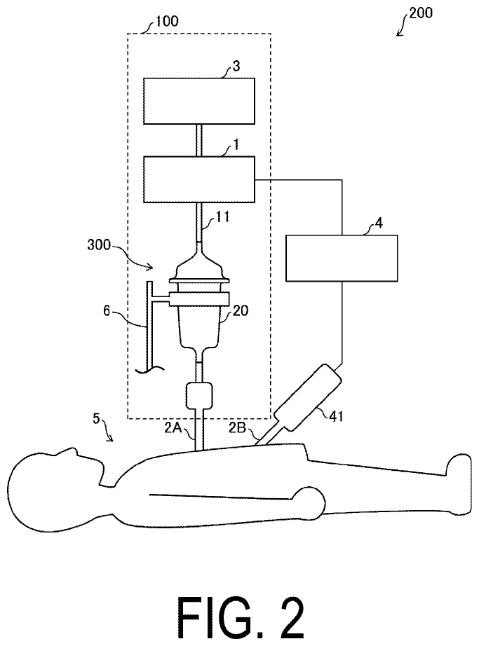

First, a surgical system 200 using a smoke evacuation device 1 according to an aspect of the present disclosure will be described with reference to FIG. 2. FIG. 2 is a schematic view of the surgical system 200 including a smoke evacuation system 100 including the smoke evacuation device 1 (intake amount adjustment mechanism) according to one aspect of the present disclosure.

The surgical system 200 is a system used when an endoscopic surgery is performed on a patient 5, and includes the smoke evacuation system 100 and a cautery device 4. The smoke evacuation system 100 is a system for discharging surgical smoke staying in an abdominal cavity of the patient 5, and includes a smoke evacuation tube set 300, the smoke evacuation device 1, and a suction device 3. In the present description, a gas including surgical smoke and moisture is simply referred to as a gas.

The smoke evacuation tube set 300 is a mechanism forming a channel for a gas from a trocar 2A disposed on an abdominal wall of the patient 5 to the suction device 3, and includes a tube 11 and a water trap 20.

The trocar 2A is a tubular member, and is a medical instrument that is punctured into an abdominal region of the patient 5, secures a path between the abdominal cavity of the patient 5 and the outside of the body of the patient 5, and functions as a guide tube. In particular, the trocar 2A is indwelled in the body wall to ensure a smoke evacuation path for the surgical smoke. The trocar 2A is connected to the tube 11 to form a portion of a gas channel from the abdominal cavity of the patient 5 to the outside of the body of the patient 5.

The tube 11 is a tube made of an elastically deformable material. The tube 11 may be a tube generally used in medical practice. As the tube 11, for example, a silicon tube having an inner diameter of 5 mm is used, but the material and the tube diameter of the tube 11 are not particularly limited.

As will be described in detail below, the water trap 20 is a medical water trap that has an intake port and a discharge port, and removes at least a portion of moisture from a gas flowing from the intake port to the discharge port. The detailed structure of the water trap 20 and the principle of removing moisture from the gas passing through the water trap 20 will be described below.

As illustrated in FIG. 2, the surgical system 200 according to the present embodiment may further include a water trap holder 6 holding the water trap 20. Here, the water trap holder 6 may hold the water trap 20 at an angle at which the discharge port of the water trap 20 is located above the intake port.

In the present embodiment, the smoke evacuation tube set 300 includes a plurality of the tubes 11. To be more specific, one of the tubes 11 is connected to the trocar 2A and to the intake port of the water trap 20, and another tube 11 is connected to the discharge port of the water trap 20 and to the suction device 3. In other words, the tubes 11 form a gas channel from the trocar 2A to the suction device 3 via the water trap 20.

The smoke evacuation device 1 is a device adjusting an amount of the gas sucked by the suction device 3 by changing the inner diameter of the channel formed by the tube 11. The smoke evacuation device 1 is disposed in a portion of the tube 11 between the water trap 20 and the suction device 3, and opens and closes the channel by, for example, pressing the tube 11.

The suction device 3 is a device sucking a gas in the abdominal cavity of the patient 5. The suction device 3 may be a device having a suction function that is generally used in medical practice. As the suction device 3, for example, a medical gas piping facility which is connected to a suction facility by connecting the tube 11 to a piping terminal installed on a wall surface of an operating room may be used.

The cautery device 4 is a medical cautery device including a surgical cautery instrument 41 used for endoscopic surgery. Examples of the surgical cautery instrument 41 include an ultrasonic scalpel, an electric scalpel, a laser scalpel, and cauterizing forceps. The treatment on a body tissue of the patient 5 by the surgical cautery instrument 41 may be performed via, for example, a trocar 2B different from the trocar 2A among trocars placed in the body wall of the patient 5. The trocar 2B may be a member having the same shape and the same material as those of the trocar 2A.

### Operation of Surgical System

An operation of the surgical system 200 when the surgical system 200 is used to perform surgery on the patient 5 will be described.

In general, in a laparoscopic surgery, the abdominal cavity of the patient 5 may be provided with not only a gas including nitrogen or the like provided to secure the atmospheric pressure of the abdominal cavity and to secure the surgical field, but also the surgical smoke generated by cauterization or the like of the body tissue by the surgical cautery instrument 41. The staying of the surgical smoke in the abdominal cavity of the patient 5 may lead to obstruction of a surgical field of view. Thus, the surgical smoke may be evacuated from the abdominal cavity.

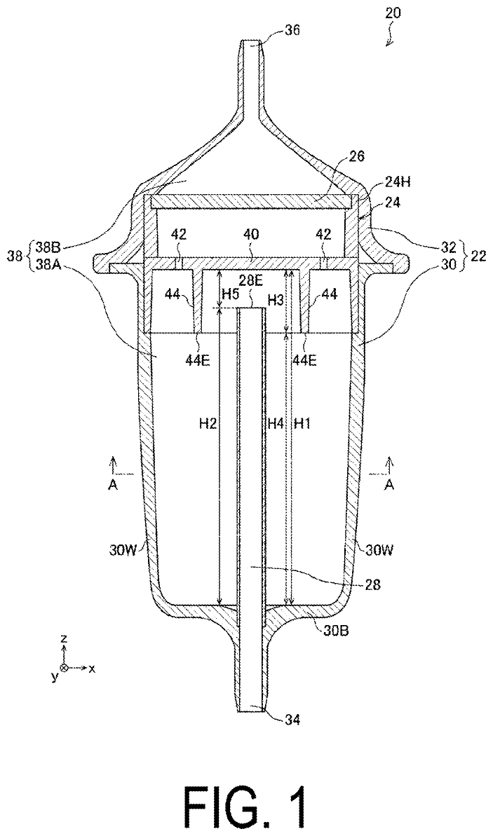

The smoke evacuation system 100 can discharge some of the gas in the abdominal cavity of the patient 5, including the surgical smoke to the outside of the body of the patient 5 by the suction of the gas by the suction device 3. Here, since the surgical smoke is a gas generated by cauterization of the body tissue of the patient 5, the surgical smoke generally includes moisture. If the moisture included in the surgical smoke infiltrates into the filter 26 illustrated in FIG. 1, the filter 26 may be clogged, and the suction of the gas by the suction device 3 may be hindered. Therefore, the smoke evacuation system 100 may include a mechanism for reducing the moisture content of the gas in the channel for the gas from the abdominal cavity of the patient 5 to the filter 26.

In the present embodiment, the smoke evacuation tube set 300 forming the gas channel from the abdominal cavity of the patient 5 to the suction device 3 includes the water trap 20 formed in the middle of the channel. Therefore, at least some of the moisture included in the surgical smoke is removed by the water trap 20.

Therefore, the smoke evacuation system 100 can reduce the moisture content of the gas adhering to the filter 26. Therefore, in the laparoscopic surgery of the patient 5, the surgical system 200 can remove moisture from the gas while discharging the gas including the surgical smoke generated in the abdominal cavity of the patient 5 to the outside of the body of the patient 5.

For example, the suction device 3 may always have a negative pressure to suck a gas. In this case, the smoke evacuation device 1 may normally close the tube 11 forming the channel for the gas from the water trap 20 to the suction device 3, and open the tube 11 only when the gas in the abdominal cavity including the surgical smoke is to be sucked.

The smoke evacuation device 1 may adjust the amount of the gas sucked by the suction device 3 in conjunction with the operation of the surgical cautery instrument 41. To be more specific, for example, the cautery device 4 may be communicatively connected to the smoke evacuation device 1, and the cautery device 4 may include a sensor for sensing cauterization of the body tissue of the patient 5 by the surgical cautery instrument 41. For example, the cautery device 4 may include a thermal sensor, and the thermal sensor may sense the movement of the surgical cautery instrument 41 by sensing the heat generated by the cauterization of the body tissue of the patient 5 by the surgical cautery instrument 41.

With the above configuration, the sensor included in the cautery device 4 senses the operation of the surgical cautery instrument 41, and the cautery device 4 controls the operation of the smoke evacuation device 1 according to the information on the operation of the surgical cautery instrument 41 sensed by the sensor. Thus, the cautery device 4 can control the operation of the smoke evacuation device 1 in conjunction with the operation of the surgical cautery instrument 41. Accordingly, for example, the surgical system 200 can perform the suction of the gas in the abdominal cavity of the patient 5 by the suction device 3 only when the cauterization of the body tissue of the patient 5 occurs, which may cause the surgical smoke in the abdominal cavity of the patient 5.

### Water Trap

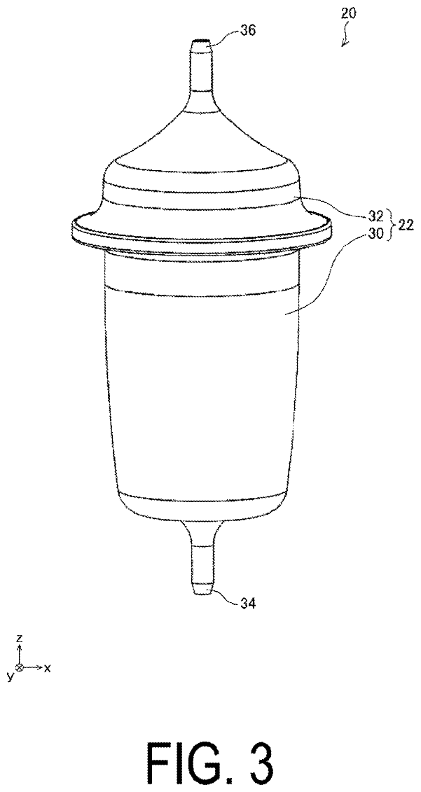

Next, the water trap 20 according to the present embodiment will be described in more detail with reference to FIGS. 1 and 3. FIG. 3 is a schematic perspective view of a substantially front side of the water trap 20 according to the present embodiment, and is a schematic view illustrating an outer shape of the water trap 20. FIG. 1 is a cross-sectional side view of the water trap 20 illustrated in FIG. 3. FIG. 1 illustrates a cross-section in a plane with a longitudinal axis of an inner cylinder 28 and extending through an opening 42, which will be described in more detail below.

The cross-sectional views of the present description including FIG. 1 illustrate not only members located in the cross-section but also members located on the back side of the cross-section with respect to the plane of the drawing. In addition, members located in the cross-section are hatched.

As illustrated in FIG. 1, the water trap 20 includes a container 22 for storing trapped moisture, a partition wall 40 provided inside the container 22 and having the opening 42, and the inner cylinder 28. The water trap 20 also includes a filter holder 24 and a filter 26.

As illustrated in FIGS. 1 and 3, the container 22 includes a first container 30 and a second container 32. For example, the container 22 may be formed by bringing the first container 30 and the second container 32 into pressure contact with each other. The first container 30 include an intake port 34 formed and the second container 32 include a discharge port 36 formed. The first container 30 and the second container 32 form an internal space 38 of the container 22. As described above, the internal space 38 of the container 22 communicates with the outside of the water trap 20 via each of the intake port 34 and the discharge port 36. The first container 30 includes a bottom portion 30B on the intake port 34 side and a sidewall 30W erected on a peripheral end portion of the bottom portion 30B.

The above-described tube 11 communicating with the trocar 2A is connected to the intake port 34. Therefore, the abdominal cavity of the patient 5 and the intake port 34 communicate with each other via the trocar 2A and the tube 11. The above-described tube 11 forming the channel from the water trap 20 to the suction device 3 is connected to the discharge port 36. Therefore, the discharge port 36 and the suction device 3 communicate with each other via the tube 11. Therefore, as described above, a gas channel is formed from the abdominal cavity of the patient 5 to the suction device 3 via the water trap 20.

Referring back to FIG. 1, in the present embodiment, the inner portion of the container 22 includes the partition wall 40. As illustrated in FIG. 1, the partition wall 40 divides the internal space 38 of the container 22 into a first space 38A on the intake port 34 side (a space on the intake port side) and a second space 38B on the discharge port 36 side (a space on the discharge port side). As described above, the first space 38A is a region surrounded by the bottom portion 30B, the sidewall 30W, and the partition wall 40. The partition wall 40 also includes at least one opening 42. Therefore, the first space 38A and the second space 38B communicate with each other via the opening 42.

The second space 38B includes filter holder 24 disposed, and the filter holder 24 and the partition wall 40 may be integrally formed. The filter holder 24 is a member supporting the filter 26, which will be described in detail below, inside the container 22.

The partition wall 40 includes a projecting portion 44. As illustrated in FIG. 1, the projecting portion 44 projects from the partition wall 40 toward the intake port 34. In the present embodiment, the projecting portion 44 projects in a substantially perpendicular direction with respect to the partition wall 40. In addition, the filter holder 24 is further formed with a protrusion portion 24H. As illustrated in FIG. 1, the protrusion portion 24H protrudes from the filter holder 24 toward the discharge port 36 side. In the present embodiment, the protrusion portion 24H protrudes from the filter holder 24, but is not limited to the configuration described above. For example, the protrusion portion 24H may be formed to protrude from any position of the inner surface of the second space 38B toward the discharge port 36 side.

Each of the first container 30, the second container 32, the partition wall 40, and the filter holder 24 described above may be formed by a method such as casting in which a flowable material is poured into a mold, then solidified, and removed from the mold.

The filter 26 is a member filtering a gas passing from the intake port 34 to the discharge port 36. In other words, the filter 26 is used to remove at least some of particulates from the gas including the particulates and passing through the filter 26. A known gas filter in the related art can be applied to the filter 26. The filter holder 24 holds the filter 26 in the second space 38B of the internal space 38 of the container 22. In other words, the container 22 contains the filter 26 disposed closer to the discharge port 36 than the partition wall 40. For example, the filter 26 may be fixed to the second space 38B by being sandwiched between the filter holder 24 and the second container 32.

In particular, a peripheral portion of the filter 26 may be fixed to the second space 38B by being sandwiched between the protrusion portion side 24H and the inner surface of the second space 38B. The peripheral edge portion of the filter 26 and the inner wall of the second space 38 may be in contact with each other without a gap to reduce the flow of the gas without passing through the filter 26. With the above-described configuration, the filter 26 is more firmly fixed, and can more effectively collect the particulates from the gas that has passed through the opening 42 and allow the particulates to flow to the discharge port 36 side.

The first space 38A of the internal space 38 in the container 22 includes the inner cylinder 28 formed. The inner cylinder 28 communicates with the intake port 34. The inner cylinder 28 extends toward the partition wall 40 in the first space 38A. However, the inner cylinder 28 is not in contact with the partition wall 40, and an end portion 28E of the inner cylinder 28 on a side opposite to the intake port 34 faces the partition wall 40 with a distance therebetween. Therefore, the gas flowing into the container 22 from the intake port 34 passes through the inner cylinder 28 and flows into the first space 38A from the end portion 28E toward the partition wall 40.

An extending direction of the inner cylinder 28, in other words, a longitudinal axis direction of the inner cylinder 28 may substantially coincide with the direction from the intake port 34 toward the discharge port 36. The longitudinal axis direction of the inner cylinder 28 may be substantially perpendicular to the partition wall 40. For example, the inner cylinder 28 may be inserted into the intake port 34 and may be fixed by fitting the inner cylinder 28 and the intake port 34 to each other. In particular, the longitudinal axis of the inner cylinder 28 may substantially coincide with the central axis of the inner cylinder 28.

### Positional Relationship Between Inner Cylinder, Projecting Portion, and Opening

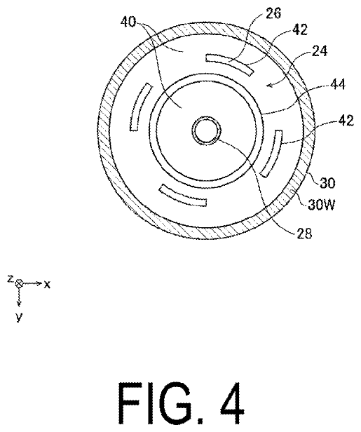

Here, with reference to FIG. 4, the positional relationship between the inner cylinder 28 and each portion of the partition wall 40 will be described in more detail. FIG. 4 is a cross-sectional view taken along the line A-A in a direction of arrow illustrated in FIG. 1, in other words, a view of a cross-section in a plane which crosses the inner cylinder 28 and is perpendicular to the longitudinal axis direction of the inner cylinder 28, in other words, the central axis direction, as viewed from the intake port 34 side.

As illustrated in FIG. 4, the projecting portion 44 is formed to surround the inner cylinder 28 when the partition wall 40 is viewed from the intake port 34 side in a plan view. For example, as illustrated in FIG. 4, the projecting portion 44 may project from a substantially circular position on the partition wall 40 in a plan view of the partition wall 40 from the intake port 34 side. However, no such limitation is intended, and for example, the projecting portion 44 may project from a position to be in a rectangular shape on the partition wall 40 in a plan view of the partition wall 40 from the intake port 34 side.

As illustrated in FIG. 4, the opening 42 is formed in the partition wall 40 closer to the wall surface side of the sidewall 30W of the first container 30 than the projecting portion 44.

A number of the openings 42 is not limited as long as at least one opening 42 is formed, and a plurality of the openings 42 may be formed. Since the partition wall 40 includes the plurality of openings 42, the gas flowing from the first space 38A into the second space 38B through the openings 42 can be suppressed from intensively passing through the vicinity of one point of the filter 26. With the above configuration, the filter 26 can be used more efficiently, and the life time of the filter 26 is prolonged.

The shape of the opening 42 may be any of various shapes such as a circular shape in addition to the fan shape illustrated in FIG. 4. In the present embodiment, the opening 42 may be formed by forming the partition wall 40 closer to the sidewall 30W of the first container 30 than the projecting portion 44 in a mesh shape. Since the water trap 20 includes the opening 42 in a mesh shape, the opening 42 can be provided with a function of a filter removing foreign matters from the gas passing through the opening 42.

The sum of opening areas of the openings 42 may be larger than the opening area of the inner cylinder 28 at the end portion 28E. According to the above-described configuration, the flow rate of the gas flowing into the first space 38A from the intake port 34 via the inner cylinder 28 becomes relatively great, and the flowing gas may easily be in contact with the partition wall 40 and the projecting portion 44. Therefore, moisture can be more efficiently removed from the gas by a mechanism for removing moisture by the water trap described below.

### Mechanism of Moisture Removal by Water Trap

Next, the principle of removing moisture from the gas passing through the inside of the container 22 from the intake port 34 to the discharge port 36 by the water trap 20 will be described. In the present embodiment, during the operation of the surgical system 200, the water trap 20 is always in a state in which the intake port 34 is maintained at the lowermost position and the discharge port 36 is maintained at the uppermost position.

As described above, the intake port 34 is connected to the tube 11 connected to the trocar 2A, and the discharge port 36 is connected to the tube 11 connected to the suction device 3. Therefore, the gas in the abdominal cavity of the patient 5 sucked by the suction device 3, which includes the surgical smoke, enters the inside of the container 22 from the intake port 34 and is discharged to the outside of the container 22 from the discharge port 36.

The gas entering the inside of the container 22 from the intake port 34 first flows toward the end portion 28E inside the inner cylinder 28. Here, since the inner cylinder 28 extends toward the partition wall 40, the gas flowing through the inner cylinder 28 is discharged from the end portion 28E toward the partition wall 40 into the first space 38A.

The gas in the first space 38A is sucked toward the second space 38B through the opening 42 formed in the partition wall 40. However, the opening 42 is located closer to the wall surface side of the sidewall 30W than the projecting portion 44 projecting from the partition wall 40 toward the intake port 34 side. When the partition wall 40 is viewed from the intake port 34 side in a plan view, since the projecting portion 44 is formed at a position surrounding the inner cylinder 28, the projecting portion 44 is located closer to the wall surface side of the sidewall 30W than the inner cylinder 28.

Due to the above-described positional relationship, intrusion of at least some of the gas discharged from the end portion 28E is obstructed by the partition wall 40 and the projecting portion 44. As a result, the gas does not flow straight to the opening 42. Instead the gas flows through the region of the first space 38A surrounded by the projecting portion 44 toward the bottom portion 30B, and then flows toward the opening 42 on the sidewall 30W side from the projecting portion 44 of the first space 38A. Therefore, at least a portion of the gas discharged from the end portion 28E stays around the projecting portion 44 for a longer time as compared with a case where the projecting portion 44 is not formed.

When the gas staying around the protruding portion 44 comes into contact with the partition wall 40, the projecting portion 44, the sidewall 30W, and the like, heat of the gas is conducted to members with which the gas comes into contact. Therefore, the temperature of the gas decreases, and the amount of saturated water vapor of the gas decreases. Therefore, moisture included in the gas in contact with the partition wall 40, the projecting portion 44, the sidewall 30W, and the like is condensed to form water droplets, and the moisture is removed from the gas. The condensed water droplets adhere to the partition wall 40, the projecting portion 44, the sidewall 30W, and the like, and then are stored, that is, trapped in the bottom portion 30B by gravity. On a route from the abdominal cavity of the patient 5 to the end portion 28E, the moisture that has already been liquefied (turned into water droplets) is also trapped in the water trap 20 by the same principle as that described above.

The gas that has entered the second space 38B from the first space 38A through the opening 42 passes through the filter 26 and is then discharged from the discharge port 36 to the outside of the water trap 20. As the gas passes through the filter 26, the filter 26 removes at least one substance from the gas. In particular, in the present embodiment, the filter 26 may be a gas filter removing particulates in the gas. With the above configuration, the filter 26 more efficiently cleans the gas passing through the water trap 20.

As described above, the water trap 20 can reduce the moisture of the gas passing through the inside the water trap 20.

### Moisture Storage in Water Trap

The moisture removed from the gas discharged from the end portion 28E and attached to the partition wall 40, the projecting portion 44, the sidewall 30W, and the like falls or flows toward the bottom portion 30B and is stored outside the inner cylinder 28 in the first space 38A. Therefore, when the smoke evacuation tube set 300 is continuously used, moisture continues to be stored in the first space 38A, and the liquid level of the moisture rises.

Here, as illustrated in FIG. 1, a height from the bottom portion 30B to the partition wall 40 is referred to as H1, and a height from the bottom portion 30B to the end portion 28E of the inner cylinder 28 on the partition wall 40 side is referred to as H2. A height from the partition wall 40 to an end portion 44E on the bottom portion 30B side of the projecting portion 44 is referred to as H3, and a height from the bottom portion 30B to the end portion 44E is referred to as H4. A height from the partition wall 40 to an end portion 28E of the inner cylinder 28 is referred to as H5. In other words, the H3 is equal to a distance by which the projecting portion 44 projects from the partition wall 40, and the H5 is equal to a distance from the end portion 28E to the partition wall 40.

In the present embodiment, it is necessary to replace the water trap 20 or remove the moisture from the water trap 20 before the liquid level of the moisture stored in the first space 38A reaches the H4. In other words, the water trap 20 can be continuously used until the liquid level of the moisture stored in the first space 38A reaches the H4.

In the present embodiment, in order for the gas to be discharged from the end portion 28E of the inner cylinder 28 toward the partition wall 40, the end portion 28E needs not to abut against the partition wall 40, in other words, the H2 may be slightly lower than the Hl.

Therefore, in the present embodiment, the ratio of the maximum volume of moisture that can be stored in the first space 38A to the total volume of the internal space 38 greatly depends on the height of the H2 and the H3 with respect to the Hl. Therefore, the water trap 20 according to the present embodiment can increase the ratio of the maximum volume of the moisture that can be stored in the first space 38A to the entire volume of the internal space 38. To be more specific, in the present embodiment, the ratio can be easily increased by making the H2 as close to the H1 as possible and making the H3 as low as possible.

In the present embodiment, the H2 is longer than the H4. In other words, the H3 is longer than the H5. Therefore, as illustrated in FIG. 1, the end portion 28E of the inner cylinder 28 is located closer to the partition wall 40 than the end portion 44E of the projecting portion 44 is. In other words, the projecting portion 44 is formed to surround the periphery of the end portion 28E of the inner cylinder 28. With this configuration, the gas discharged from the inner cylinder 28 toward the partition wall 40 more easily comes into contact with the partition wall 40 and the projecting portion 44, and thus the moisture is more efficiently removed from the gas.

When the H2 is higher than the H4 and the liquid level of the moisture stored in the first space 38A becomes higher than the H4, the liquid level comes into contact with the end portion 44E of the projecting portion 44. In this state, by the stored moisture, the gas in the first space 38A is separated into the gas on the inner cylinder 28 side with respect to the projecting portion 44 and the gas on the sidewall 30W side with respect to the projecting portion 44.

However, even in this state, the gas discharged from the inner cylinder 28 leaks to the sidewall 30W side beyond the projecting portion 44, and thus the suction of the gas can be continued. However, in the above-described state, when the gas discharged from the inner cylinder 28 flows toward the sidewall 30W from the projecting portion 44, the flowing gas becomes bubbles and moves to the sidewall 30W. Here, water droplets generated by bursting of the bubbles may adhere to the filter 26. Therefore, when the liquid level of the moisture stored in the first space 38A becomes higher than the H4, clogging of the filter 26 is likely to occur.

Therefore, the liquid level of the moisture stored in the first space 38A is desirably lower than the H4.

In the present embodiment, since the projecting portion 44 is formed on the partition wall 40, the gas in the first space 38A is easily stayed. Therefore, the moisture can be more efficiently removed from the gas passing through the water trap 20 by the projecting portion 44.

As described above, the water trap 20 according to the present embodiment can increase the ratio of the maximum volume of moisture that can be stored in the first space 38A to the entire volume of the internal space 38 while efficiently removing moisture from the passing gas.

### APPENDIX

In the present embodiment, at least one of the partition wall 40 and/or the projecting portion 44 may include a material having higher thermal conductivity than the material of the container 22 and the inner cylinder 28. With this configuration, the moisture in the gas is easily aggregated by the partition wall 40 and the projecting portion 44, thereby improving the efficiency of moisture removal from the gas by the water trap 20.

In the present embodiment, as described above, the filter 26 is disposed inside the container 22 and closer to the discharge port 36 side than the partition wall 40. In other words, the filter 26 is disposed in the second space 38B. With this configuration, in the present embodiment, the gas from which moisture has been removed in the first space 38A passes through the filter 26. For this reason, in the present embodiment, the gas containing a large amount of moisture from passing through the filter 26 can be suppressed, thereby suppressing the shortening of the life time of the filter 26.

In the present embodiment, the filter 26 and the partition wall 40 may not be in contact with each other but may be separated from each other. In other words, the filter 26 and the opening 42 may be spaced apart from each other. With this configuration, the gas that has entered the second space 38B from the first space 38A through the opening 42 is temporarily stored in the space between the filter 26 and the partition wall 40. Therefore, in the present embodiment, compared to a case where the filter 26 and the partition wall 40 are in close contact with each other, the gas from the opening 42 passes through the filter 26 more uniformly, thereby further suppressing the shortening of the life time of the filter 26. In particular, since the filter 26 is fixed by the protrusion portion 24H and the inner wall of the second space 38B, the filter 26 and the partition wall 40 can be more efficiently separated from each other.

With the above-described configuration, shortening of the life time of the filter 26 is suppressed, and thus the cycle of discarding the filter 26 is prolonged, and an increase in the amount of wastes generated by use of the water trap 20 is suppressed. Therefore, the above configuration can contribute to achievement of sustainable development goals (SDGs).

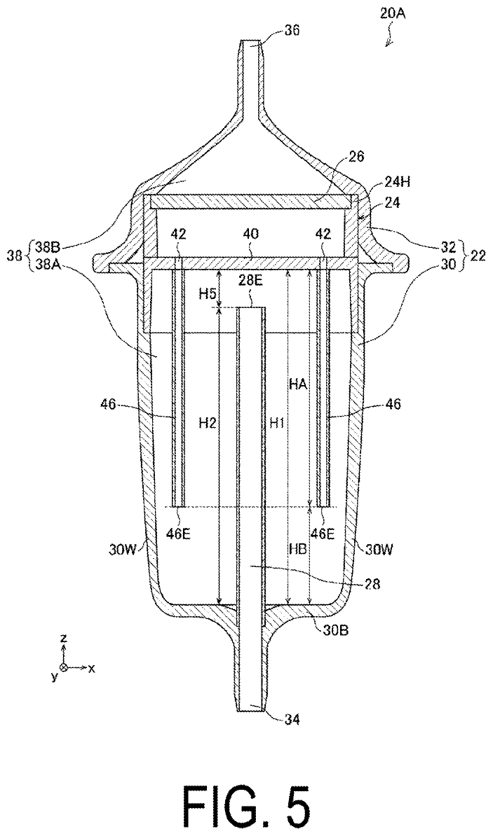

Comparative Embodiment In order to describe the effect of the water trap 20 according to the present embodiment in more detail, a water trap according to a comparative embodiment corresponding to the present embodiment will be described with reference to FIG. 5.

As compared with the water trap 20 according to the present embodiment, a water trap 20A according to the comparative embodiment includes a second inner cylinder 46 that communicates with one of the openings 42 and extends toward the bottom portion 30B in the first space 38A, instead of the projecting portion 44. For example, the second inner cylinder 46 is formed for each opening 42. Therefore, as compared with the partition wall 40 according to the present embodiment, in the partition wall 40 according to the comparative embodiment, the opening 42 is not formed on the sidewall 30W side with respect to the second inner cylinder 46 protruding from the partition wall 40.

Except for the feature described above, the water trap 20A according to the comparative embodiment has the same configuration as the water trap 20 according to the present embodiment.

When the water trap 20A according to the comparative embodiment is applied to the surgical system 200, at least some of moisture is removed from the gas in the abdominal cavity of the patient 5 by some of the gas passing through the water trap 20A from the intake port 34 to the discharge port 36. To be more specific, the gas discharged from the end portion 28E of the inner cylinder 28 to the first space 38A passes through the second inner cylinder 46, then passes through the opening 42, and is discharged to the second space 38B.

Therefore, the gas discharged from the end portion 28E to the first space 38A advances toward an end portion 46E on the bottom portion 30B side of the second inner cylinder 46, in other words, toward the bottom portion 30B side. Therefore, the gas discharged from the end portion 28E to the first space 38A stays around the inner cylinder 28 and the second inner cylinder 46 for a long time, and moisture is removed from the gas in contact with the inner cylinder 28, the sidewall 30W, the second inner cylinder 46, and the like. The moisture falls or flows toward the bottom portion 30B and is stored in the first space 38A.

Here, as illustrated in FIG. 5, a height from the partition wall 40 to the end portion 46E is referred to as HA, and a height from the bottom portion 30B to the end portion 46E is referred to as HB.

In the water trap 20A according to the comparative embodiment, in order to efficiently remove moisture from the gas, it is necessary to increase the distance from the end portion 28E to the end portion 46E so that the gas discharged from the end portion 28E remains in the first space 38A for a long time. Therefore, in the water trap 20A according to the comparative embodiment, in order to efficiently remove moisture from the gas, it is necessary to increase the height from the partition wall 40 to the end portion 46E, in other words, HA. However, when HA increases, the height from the bottom portion 30B to the end portion 46E, in other words, HB decreases.

As described above, when the water trap 20A is used, moisture is stored in the first space 38A. Therefore, when the liquid level of the moisture stored in the first space 38A reaches HB, the second inner cylinder 46 is blocked by the moisture. When the second inner cylinder 46 is blocked, the gas from the intake port 34 to the discharge port 36 can be continuously passed. Therefore, when HB is low, the ratio of the maximum volume of moisture that can be stored in the first space 38A to the total volume of the first space 38A decreases.

In the water trap 20A according to the comparative embodiment, it is necessary to increase HB in order to attain the ratio of the maximum volume of the moisture storable in the first space 38A. However, in the comparative embodiment, increasing HB is synonymous with decreasing HA, and thus is synonymous with decreasing the effectiveness of removing moisture from the gas by the water trap 20A.

In particular, in order for the water trap 20A according to the comparative embodiment to ensure the same proportion of the maximum volume of moisture that can be stored in the first space 38A as that of the water trap 20 according to the present embodiment, HB needs to be greater than H2. However, when HB exceeds the H2, the gas discharged from the end portion 46E may directly proceed toward the end portion 46E without proceeding toward the bottom portion 30B. Therefore, this configuration is not a configuration enabling efficient removal of moisture from the gas.

As described above, it is difficult for the water trap 20A according to the comparative embodiment to achieve both an increase in the moisture content of the moisture that can be stored and an increase in the moisture content of the moisture that can be removed from the gas, compared to the water trap 20 according to the present embodiment.

On the other hand, in the water trap 20 according to the present embodiment, the moisture content of the storable moisture greatly depends on the H2, and even if the H3 is increased in order to efficiently remove moisture from the gas, the storable moisture content is not greatly affected. Therefore, the water trap 20 according to the present embodiment can increase the ratio of the maximum volume of moisture that can be stored in the first space 38A to the entire volume of the internal space 38 while efficiently removing moisture from the passing gas.

Second Embodiment

### Other Examples of Projecting Portion

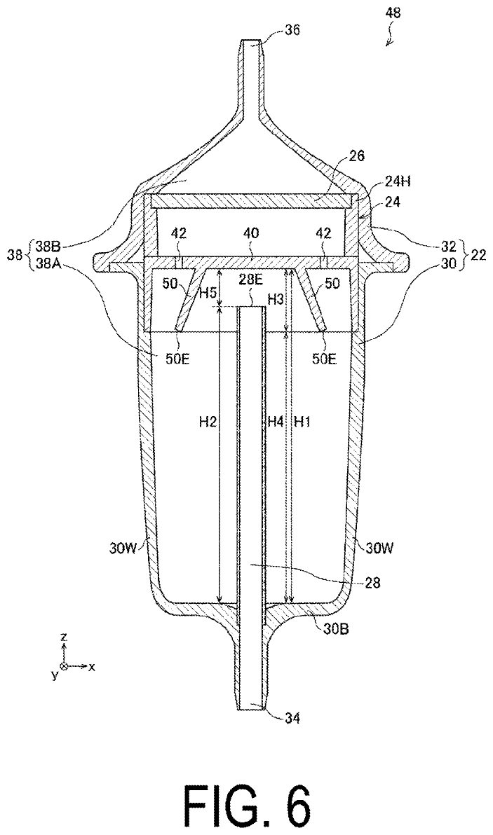

FIG. 6 is a side cross-sectional view of a water trap 48 according to the present embodiment, illustrating a cross-section corresponding to FIG. 1. In the present description, each member having the same function is denoted by the same name and assigned with the same reference sign, and explanation is not repeated as long as there is no difference in the configuration.

The water trap 48 according to the present embodiment has the same configuration as that of the water trap 20 according to the previous embodiment except that a projecting portion 50 is provided instead of the projecting portion 44. The projecting portion 50 projects from the partition wall 40 toward the intake port 34. Here, in the present embodiment, the projecting portion 50 projects in a direction that is inclined from the perpendicular with respect to the partition wall 40.

To be more specific, as illustrated in FIG. 6, the projecting portion 50 may project in a direction in which an end portion 50E on the bottom portion 30B side is closer to the sidewall 30W side as compared with a case where the projecting portion 50 projects in a direction perpendicular to the partition wall 40. However, no such limitation is intended, and the projecting portion 50 may project in a direction in which the end portion 50E on the bottom portion 30B side is closer to the inner cylinder 28 side as compared with a case where the projecting portion 50 projects in a direction perpendicular to the partition wall 40.

The water trap 48 according to the present embodiment achieves the same effect as the water trap 20 according to the previous embodiment for the same reason as described in the previous embodiment. In addition, in the present embodiment, when the height from the partition wall 40 to the end portion 50E is the same as the H3 described in the previous embodiment, the surface area of the projecting portion 50 is larger than the surface area of the projecting portion 44. Therefore, the water trap 48 according to the present embodiment can more efficiently remove moisture from gas in the projecting portion 50 without changing the height of the projecting portion 50 projecting from the partition wall 40.

The water trap 48 according to the present embodiment can be applied to the smoke evacuation tube set 300, like the water trap 20 according to the previous embodiment. The smoke evacuation tube set 300 provided with the water trap 48 according to the present embodiment can be applied to the surgical system 200.

### Third Embodiment

### Example of Projecting Portion Having Tapered Shape

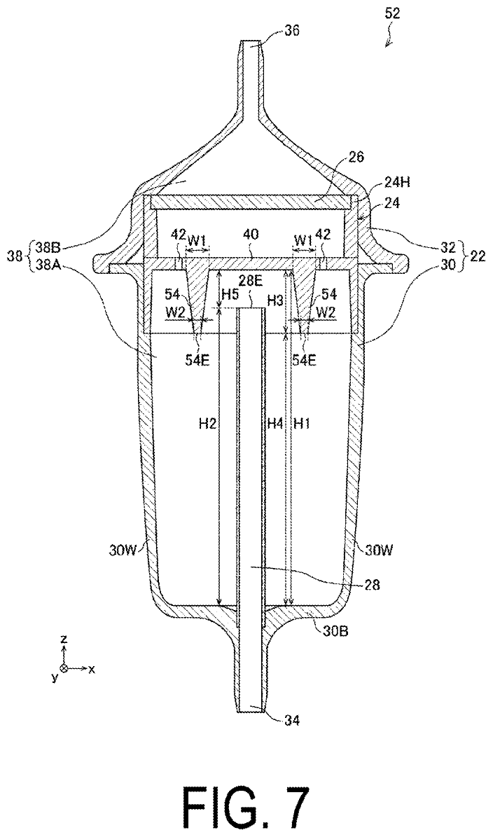

FIG. 7 is a side cross-sectional view of a water trap 52 according to the present embodiment, illustrating a cross-section corresponding to FIG. 1. The water trap 52 according to the present embodiment has the same configuration as that of the water trap 20 according to the first embodiment except that a projecting portion 54 is provided instead of the projecting portion 44.

The projecting portion 54 projects from the partition wall 40 toward the intake port 34. Here, in the present embodiment, the projecting portion 54 is formed such that the width of the projecting portion 54 in the direction from the inner cylinder 28 toward the sidewall 30W gradually increases from an end portion 54E on the intake port 34 side toward the partition wall 40 side.

In FIG. 7, a width of the projecting portion 54 on the partition wall 40 side in the direction from the inner cylinder 28 toward the sidewall 30W is referred to as W1, and a width of the end portion 54E in the direction from the inner cylinder 28 toward the sidewall 30W is referred to as W2. In the present embodiment, the W1 is larger than the W2. The width of the projecting portions 54 in the direction from the inner cylinder 28 toward the sidewall 30W gradually increases from the end portion 54E to the sidewall 30W from the W2 to the W1. Therefore, in a cross-sectional view taken along the central axis of the inner cylinder 28, the width of the projecting portion 54 on the partition wall 40 side is larger than the width on the intake port 34 side.

The water trap 52 according to the present embodiment achieves the same effect as the water trap according to each of the above-described embodiments for the same reason as that described in the first embodiment. In addition, in the present embodiment, when the height from the partition wall 40 to the end portion 54E is the same as the above-described H3, the surface area of the projecting portion 54 is larger than the surface area of the projecting portion 44. Therefore, for the reason described in the previous embodiment, the water trap 52 according to the present embodiment can more efficiently remove moisture from the gas in the projecting portion 54 without changing the height of the projecting portion 54 projecting from the partition wall 40.

The filter holder 24 of the water trap 52, which is formed integrally with the partition wall 40, can be formed more easily by molding. More specifically, when the partition wall 40 and the filter holder 24 are integrally formed by a mold, the solidified partition wall 40 and filter holder 24 can be easily removed from the mold in a direction from the end portion 54E of the projecting portion 54 toward the partition wall 40, thereby further facilitating the manufacturing.

In the present disclosure, the invention has been described above based on the various drawings and examples. However, the invention according to the present disclosure is not limited to each embodiment described above. That is, the embodiments of the invention according to the present disclosure can be modified in various ways within the scope illustrated in the present disclosure, and embodiments obtained by appropriately combining the technical means disclosed in different embodiments are also included in the technical scope of the invention according to the present disclosure. In other words, note that a person skilled in the art can easily make various variations or modifications based on the present disclosure. Note that these variations or modifications are included within the scope of the present disclosure.

For example, the projecting portion of the water trap according to the present disclosure may have a shape obtained by combining the shape of the projecting portion 50 and the shape of the projecting portion 54 described above. To be more specific, the projecting portion according to the present disclosure may project in a direction inclined from the perpendicular with respect to the partition wall 40, and in a cross-sectional view along the central axis of the inner cylinder 28, the width on the partition wall 40 side may be larger than the width on the intake port 34 side.

### REFERENCE SIGNS

20, 48, 52 Water trap

22 Container

26 Filter

28 Inner cylinder

34 Intake port

36 Discharge port

40 Partition wall

42 Opening

44, 50, 54 Projecting portion

200 Surgical system

300 Smoke evacuation tube set

1. A medical water trap comprising:
a container comprising an intake port and a discharge port;
a partition wall configured to divide an internal space of the container into a space on an intake port side and a space on a discharge port side;
an inner cylinder configured to communicate with the intake port and to extend toward the partition wall in the space on the intake port side; and
a projecting portion configured to project from the partition wall toward the intake port side and to surround the inner cylinder in a plan view of the partition wall from the intake port side, wherein
at least one opening in the partition wall on a side of the partition wall that is closer to a wall surface of the container than to the projecting portion.

2. The medical water trap according to claim 1, further comprising
a filter disposed inside the container that is located closer to the discharge port side than to the partition wall.

3. The medical water trap according to claim 2, wherein
the filter is a gas filter that removes a particulate in a gas.

4. The water trap according to claim 2, further comprising
a filter holder on the discharge port side, wherein
the filter is held by the filter holder and an internal wall of the container on the discharge port side.

5. The medical water trap according to claim 2, wherein
the at least one opening and the filter are spaced apart from each other.

6. The medical water trap according to claim 1, wherein
a distance by which the projecting portion projects from the partition wall is longer than a distance from an end portion of the inner cylinder on a side opposite to the intake port side to the partition wall.

7. The medical water trap according to claim 1, wherein
the at least one opening is a plurality of openings in the partition wall.

8. The medical water trap according to claim 1, wherein the at least one opening is a plurality of openings that have a mesh shape.

9. The medical water trap according to claim 1, wherein
a sum of opening areas of the at least one opening is larger than an opening area of an end portion of the inner cylinder on a side opposite to the intake port side.

10. The medical water trap according to claim 1, wherein
the projecting portion projects in a direction inclined from a perpendicular with respect to the partition wall.

11. The medical water trap according to claim 1, wherein
a width of the projecting portion on a partition wall side is larger than a width of the projecting portion on the intake port side in a cross-sectional view taken along a central axis of the inner cylinder.

12. The medical water trap according to claim 1, wherein
at least one of the partition wall and/or the projecting portion comprises a material having a higher thermal conductivity than a thermal conductivity of the container and the inner cylinder.

13. A smoke evacuation tube set comprising:
the medical water trap according to claim 1; and
a tube forming a channel for a gas from a trocar to a suction device configured to suck the gas via the medical water trap.

14. A surgical system comprising:
the smoke evacuation tube set according to claim 13; and
a water trap holder configured to hold the medical water trap at an angle at which the discharge port of the medical water trap is located above the intake port.

15. The surgical system according to claim 14, further comprising:
the suction device;
a surgical cautery instrument; and
an intake amount adjustment mechanism configured to adjust an amount of a gas sucked by the suction device in conjunction with an operation of the surgical cautery instrument.
